(19) United States
(12) Patent Application Publication
(10) Pub. No.: US 2017/0319563 A1
(43) Pub. Date: Nov. 9, 2017

(54) STEROID-FREE DISEASE MANAGEMENT

(71) Applicant: Asana BioSciences, LLC, Lawrenceville, NJ (US)

(72) Inventors: Roger Astbury Smith, Chester Springs, PA (US); Nicholas James Laping, West Chester, PA (US); Sandeep Gupta, Plainsboro, NJ (US)

(21) Appl. No.: 15/329,465

(22) PCT Filed: Jul. 31, 2014
(86) PCT No.: PCT/US2014/049187

§ 371 Date: Jan. 26, 2017

(51) Int. Cl. A61K 31/4439; C07D 213/30; C07D 213/84; C07D 413/06; C07D 417/00; A61K 31/00; A61K 45/06

(52) U.S. Cl. CPC A61K 31/4439; C07D 213/84; C07D 213/30; C07D 413/06; A61K 31/00; A61K 45/06; C07D 417/00

(57) ABSTRACT

The present invention relates to the use of the compound 1-(2-(4-fluorophenyl)thiazol-5-yl)-1-(pyridin-4-yl)ethanol (“ASNOO1”) in treatments without concomitant use of a steroid and/or in the form of a racemic mixture, e.g. in prostate cancer treatment. It also relates to its use in methods that are food indifferent.

### BACKGROUND OF THE INVENTION

Prostate cancer is the most common type of cancer in the US, with 240,000 new cases per year. Currently, the initial treatment for men with metastatic disease is androgen deprivation, either by surgical castration or medical castration with androgen deprivation therapy (e.g. Lupron, Vantas). While androgens are a major driver of prostate cancer growth, prostate cancer can progress to escape standard androgen deprivation therapy by, inter alia, relying on extra-testicular CYP17 enzyme activity for androgen synthesis. This has been known to occur in, for example, the adrenal glands.

When a patient is undergoing chemotherapy, burdening them with as few additional medications as possible is a significant priority. This can reduce side effects and possible drug interactions potentially improving the patient's quality of life. Other known prostate cancer treatments, despite being potent inhibitors of 17,20-lyase, such as abiraterone acetate sold under the trademark ZYTIGA, and orteronel (TAK-700) (recently abandoned after phase III trials because it did not extend overall patient survival), require concomitant use of steroids. See Tolcher and Cooper, Castration-Resistant Prostate Cancer-Hormone Therapy Redux, J. of Clinical Oncology, Mar. 20, 2010, vol.28, no.9, 1447-1449 and Yamaoka M., et al., Orteronel(TAK-700), a novel non-steroidal 17,20-lyase inhibitor: effects on steroid synthesis in human and monkey adrenal cells and seruim steroid levels in cynomolgus monkeys; J. Steroid Biochem Mol Biol, 2012 April 129(3-5) 115-28 (e-pub Jan. 12, 2012). Designing a chemotherapy needing as few supporting medications as possible is an important aspect of treatment.

The stereochemistry of chemotherapeutic agents may also play a role in maximizing the quality of life for cancer patients. If one stereoisomer has a better therapeutic index than another, it may be possible to give a lower dose of the more active isomer reducing further the medication burden on the system. And, of course, eliminating a stereoisomer or isomers having toxicity can have a direct benefit on quality of life.

Not surprisingly, since an FDA policy statement in 1992 expressing a preference for resolved stereoisometric drugs, the number of racemic drugs brought to the market has dropped precipitously. Where they used to dominate drug approvals, they now account for only 13% of the new molecular entities approved by the FDA over the 10 years from 2002 through 2011. Agranat et al., The Predicated Demise of Racemic New Molecular Entities is an Exaggeration, Nature Reviews Drug Discovery 11, 972-973 (December 2012). The clear expectation now is that resolved stereoisomers are better drug candidates than racemic mixtures.

New and more effective treatments for humans that selectively suppress testosterone production in cancer patients are, of course, highly desirable.

### BRIEF SUMMARY OF THE INVENTION

In one aspect, the invention includes methods of treating human patients by administering to human patients in need of such treatment an effective amount of a mixture of enantiomers of 1-(2-(4-fluorophenyl)thiazol-5-yl)-1-(pyridin-4-yl)ethanol hereinafter identified as “ASN001” (elsewhere known as “EN3356”). Embodiments of these methods include treating a human patient suffering from prostate cancer. In another embodiment, the invention includes a method of treating a human patient suffering from castration-resistant prostate cancer. In yet another embodiment, the invention includes a method of reducing testosterone synthesis in a patient suffering from a cancer susceptible to testosterone deprivation. In another embodiment, the invention includes a method of treating a cancer susceptible to testosterone deprivation. In still another embodiment, the invention includes a method of selectively inhibiting testosterone synthesis in a human patient in need thereof without a similar or greater reduction in cortisol synthesis. In a further embodiment, the invention includes a method of testosterone deprivation in a human patient in need thereof.

The mixture of enantiomers may be a racemic mixture of ASN001.

The amount of ASN001 to be administered daily in each embodiment of this aspect of the invention is an effective amount which can be as little as about 10 mg as high as about 2 grams. The dose may also range from about 10 to about 500 mg. The amount administered daily may also range from about 25 to about 400 mg and alternatively, the amount administered daily ranges from about 35 to about 350 mg. In still another variation of these embodiments, the amount administered daily ranges from about 50 to about 300 mg. This amount can be administered in a single dose or up to four doses given throughout the day (“divided doses”) and is administered each day of each administration cycle upon which ASN001 is to be administered. In one embodiment, each cycle is 14 consecutive days. In another embodiment, each cycle is 30 consecutive days. In still a further embodiment, ASN001 is administered every day (or substantially every day with brief interruptions and/or rest periods as determined medically necessary and appropriate) until such time that the therapy is no longer effective or cannot be tolerated by the patient.

In a first alternative aspect, the methods of treatment identified above can each be practiced by administering orally to a human patient in need thereof a chemotherapeutic regimen consisting essentially of an effective amount of ASN001 which can range from about 10 mg to about 2 grams daily. This is advantageously accomplished without the concomitant administration of a steroid, such as prednisone. The amount administered daily may also range from about 10 to about 500 mg or from about 25 to about 400 mg and alternatively, the amount administered daily may range from about 35 to about 350 mg. In still another variation of these embodiments, the amount administered daily ranges from about 50 to about 300 mg. Whatever the amount, the ASN001 is administered each day of each administration cycle upon which ASN001 is to be administered and can be administered in a single dose or up to four doses given throughout the day. The ASN001 used may be a substantially pure enantiomer, a mixture of enantiomers, or a racemic mixture.

In another aspect, the present invention relates to a daily regimen of ASN001. The regimen is suitable for: administration to a human patient suffering from prostate cancer; administration to a human patient suffering from castration-resistant prostate cancer; administration to a human patient suffering from a cancer susceptible to testosterone deprivation; administration to a human patient suffering from a condition that can be treated by selectively inhibiting testosterone synthesis without a similar or greater reduction in cortisol synthesis; and for administration to a human patient in need of testosterone deprivation therapy. The regimen comprises about 10 mg to about 2 grams or in another embodiment from about 10 mg to about 500 mg of a mixture of enantiomers of ASN001 divided into from one to four daily doses. In one embodiment, each administration cycle is 14 consecutive days. In another embodiment, each administration cycle is 30 consecutive days.

In still a further embodiment, ASN001 is administered every day (or substantially every day with brief interruptions and/or rest periods as determined medically necessary and appropriate) until such time that the therapy is no longer effective or cannot be tolerated by the patient. Alternatively, the amount administered daily ranges from about 25 to about 400 mg and in a further alternative, the amount administered daily ranges from about 35 to about 350 mg. In still another variation, the amount administered daily ranges from about 50 to about 300 mg. In still a further embodiment of this aspect, the mixture of enantiomers administered is racemic ASN001.

In another aspect, the invention is a daily chemotherapeutic regimen of racemic ASN001 suitable for oral administration to a human patient. The dosage forms used are solid oral dosage forms and they will contain between 10 mg and 2 grams of racemic ASN001, either alone or in combination with a pharmaceutical carrier or excipient. In another embodiment, the ASN001 used may be a mixture of enantiomers. This includes a mixture in any proportion from about 95/5-5/95 by weight (referred to generally herein as simply a “mixture”). The ASN001 in the dosage form may alternatively be a substantially pure single enantiomer (greater than 95% of a single enantiomer) of ASN001.

ASN001 is a potent and selective inhibitor of CYP17 lyase and testosterone synthesis. Unlike other compounds known for inhibiting CYP17 activity, ASN001's high selectivity for inhibiting testosterone synthesis over cortisol synthesis makes ASN001 an excellent choice for treating conditions that can be impacted by suppressing testosterone synthesis such as cancers which are susceptible to androgen deprivation.

This selectivity has additional potential benefits; among them the ability to treat patients without the concomitant use of a steroid such as prednisone, dexamethasone and the like. Steroids, of course, are widely known for their anti-inflammatory properties. Steroids are also well known in a number of treatment areas including chemotherapy. However, steroids have a variety of side effects such as glaucoma, edema, high blood pressure, mood swings, weight gain, increased risk of infection, high blood sugar, and a lower wound healing. Indeed, steroids, depending on patient, the type of steroid used, the dose, etc. can impact the patient's condition when therapy is discontinued. For example, rapid discontinuance of steroid therapy has been known to cause depression. ASN001 treatments should not require concomitant use of steroids. The ability to selectively inhibit testosterone in treating patients in need thereof, without requiring the concomitant use of a steroid, is a true advance.

It has also been discovered that administering the racemic mixture of the plus (+) and minus (−) optical isomers (enantiomers) of ASN001 (also referred to herein as (+)ASN001 and (−)ASN001 respectively) is unexpectedly equal to or superior than administering an equal amount of the more active enantiomer alone i.e., in the absence of substantial amounts of the other enantiomer. When the optical isomers were independently tested in vitro, it was found that one enantiomer, the one having the negative (−) optical rotation ((−)ASN001), was significantly more potent than the other, i.e., the (+)enantiomer ((+)ASN001).

In testing the racemic mixture in comparison to an equal amount of substantially pure (−)enantiomer, one would expect that the (−)enantiomer would provide greater in vivo activity. After all, the racemic mixture includes 50% by weight of the less active (+)enantiomer. But, the racemic mixture was found to be at least as active, if not more active, than (−)enantiomer in in vivo models. Without wishing to be bound by any particular theory of operation, it is believed that there is improved pharmacokinetic disposition of the more potent (−)enantiomer in the presence of the (+)enantiomer.

This realization suggests the possible use of a lower dose of the racemic mixture, relative to the dose of either enantiomer administered alone, to achieve an equivalent therapeutic benefit. A reduction in the amount of chemotherapeutic load on the patient might reduce side effects and improve quality of life without adversely impacting treatment outcome.

### DETAILED DESCRIPTION

The term “comprising” as used herein is meant to indicate that the disclosure is “open-ended.” The use of “comprising” in connection with describing or claiming a method, regimen or dosage form in accordance with the present invention includes ASN001. It does not exclude the use of additional therapeutically active ingredients such as steroids or other forms of chemotherapy. In contrast, “consisting essentially of” as used herein is meant to exclude things that would be contrary to the basic and novel characteristics of the invention. In the context of the present invention, the use of “consisting essentially of” is meant to denote the use of ASN001 without the concomitant use of steroids, which is contrary to the basic and novel characteristics of certain aspects of the invention. The use of “consisting essentially of,” however, is not meant to exclude the concomitant use of other additional nonsteroidal chemotherapeutic or nonsteroidal therapeutically active agents.

“ASN001” as used herein refers to the compound 1-(2-(4-fluorophenyl)thiazol-5-yl)-1-(pyridin-4-yl)ethanol as well as salts, solvates, and prodrugs thereof. Unless expressly referenced or unless the context suggests otherwise, ASN001 refers to its individual enantiomers and mixtures of same in any proportion and includes all salts, solvates, and prodrugs of these. The individual enantiomers of ASN001 can be resolved using the general procedure described in, for example, Method 1 of Examples 8 and 9 of WO 2013/049559 (published in the US as United States Patent Application Publication No. US 2013/0085148).

The terms “enantiomer” and “optical isomer” are used interchangeably herein unless indicated otherwise and refer to a resolved enantiomer of ASN001 substantially free of any other enantiomer. “Substantially free” generally indicates less than 5% by weight of any unspecified or unintended enantiomer. “Mixture” as used in connection with ASN001 means a mixture of enantiomers, or more broadly, two or more stereoisomers, where no single stereoisomer is present in an amount greater than about 95% by weight. In the context of enantiomers that means that the proportions making up a “mixture” generally ranges from about 95/5 to about 5/95 by weight. A racemate or racemic mixture is one particular type of such a mixture. A racemic mixture of ASN001 can be produced as described in Example 7 of WO 2013/049559. A mixture that is 75% of the (−)enantiomer and 25% of the (+)enantiomer is another example of a “mixture.”

Salts of ASN001 include those disclosed in the aforementioned PCT application and include, but are not limited to, water-soluble and water-insoluble salts. Salts disassociate when dissolved. Examples of acids which can be used to form salts with ASN001 include, without limitation, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, trifluoroacetic, and camphorsulfonic. Any other salt former can be used as well.

The term “solvate” as used herein is a combination, physical association and/or solvation of ASN001 with a solvent molecule. This physical association involves varying degrees of ionic, covalent and hydrogen bonding. Solvates generally include solvent molecules incorporated into the crystal lattice of a crystalline solid. One common solvate is a hydrate in which molecules of ASN001 are associated with molecules of water of crystallization. Solvates can associate a fraction of a solvent molecule with each molecule of ASN001, a one-to-one association, and or an association of more than one solvent molecule with each molecule of ASN001. Common solvates include a hemisolvate (0.5 molecules of solvent per molecule of ASN001) a monosolvate (a one-to-one molecule of solvent per molecule of ASN001), a sesquisolvate (3 molecules of solvent per 2 molecules of ASN001), a pentasolvate (5 molecules of solvent per molecule of solvent), etc.

A prodrug is a form of an active substance that is typically not pharmaceutically active, or at least less pharmaceutically active than the desired active form of the drug. However, once ingested, the body metabolizes the prodrug. This means converting, by normal metabolic processes such as hydrolysis, the prodrug, to produce something that is pharmaceutically active. Abiraterone acetate is an example of a prodrug where the acetate group is cleaved during metabolism to produce abiraterone.

ASN001 may be useful in treating conditions which are associated with CPY17 activity. In one embodiment, such a disease is associated with abnormal cellular proliferation, particularly the abnormal proliferation of cells which is sensitive to hormones such as testosterone or estrogen. The term “abnormal cellular proliferation” refers to the uncontrolled growth of cells which are naturally present in a mammalian body. In one embodiment, a disease which is characterized by abnormal cellular proliferation is cancer, including, without limitation, cancer of the prostate, head, neck, eye, mouth, throat, esophagus, bronchus, larynx, pharynx, chest, bone, lung, colon, rectum, stomach, bladder, uterus, cervix, breast, ovaries, vagina, testicles, skin, thyroid, blood, lymph nodes, kidney, liver, intestines, pancreas, brain, central nervous system, adrenal gland, marrow cells, white blood cells or skin or a leukemia. In one embodiment, the disease characterized by abnormal cellular proliferation is cancer of the prostate, breast or bladder.

The term “prostate cancer” refers to a malignant tumor of the prostate, the gland that produces some of the components of semen. Prostate cancer is a disease in which cells in the prostate gland become abnormal and start to grow uncontrollably, forming malignant tumors. In the context of the present invention, treating “prostate cancer” contemplates treating human patients having prostate cancer with ASN001.

“Castration-resistant prostate cancer,” also known as castrate-recurrent prostate cancer, refers to a condition where there is prostate cancer disease progression despite androgen-deprivation therapy (ADT) which is normally accomplished by the use of luteinizing hormone releasing hormone (LHRH) or by bilateral orchiectomy. The disease progression may present as one or any combination of a continuous rise in serum levels of prostate-specific antigen (PSA), progression of pre-existing disease, or appearance of new metastases.

“A cancer susceptible to testosterone deprivation” (TD) as used herein refers to a form of cancer that can be treated by depriving it of testosterone. Such cancers include, but are not limited to, prostate cancer, bladder cancer, and breast cancer.

“Reducing testosterone synthesis in a patient suffering from a cancer susceptible to testosterone deprivation” means administering an agent to a patient in need thereof which is shown, in vitro or in vivo to cause a reduction in testosterone levels.

“Selectively inhibiting testosterone synthesis without a similar or greater reduction in cortisol synthesis” means suppression of testosterone levels to a significantly greater degree than suppression of cortisol levels. This can be determined in vitro or in vivo. In one embodiment, the relative suppression of testosterone synthesis is at least two fold greater than the relative suppression of cortisol synthesis measured in vitro as described herein.

ASN001 has one chiral center. The individual enantiomers of ASN001 are both potent and selective inhibitors of CYP17. In human testicular microsomes, for example, ASN001 and the enantiomers (+)ASN001 and (−)ASN001 were determined to have CYP17 C17,20-lyase IC₅₀ values of 15, 63 and 8 nM, respectively. Similar relative potency results for the racemate and enantiomers have also been obtained experimentally in rat testicular microsomes and human H295R adrenocortical carcinoma cells. In summary ASN001 and its individual enantiomers are all potent inhibitors of CYP17 activity and testosterone synthesis in biochemical and cellular functional assays. However, (−)ASN001 is consistently more potent in vitro than the racemic mixture, which is itself more potent than (+)ASN001.

In in vivo studies in male Sprague-Dawley (SD) rats, ASN001 and its individual enantiomers were each determined to suppress testosterone levels (compared to control animals). Treatment with 3, 10 and 30 mg/kg oral administration (PO) of enantiomer (+)ASN001 caused 59 to 87% inhibition of testosterone at 3 hours post-dosing, whereas treatment with 3, 10 and 30 mg/kg PO of enantiomer (−)ASN001 caused 73 to 80% inhibition of testosterone at 3 hours. Surprisingly, in the same study, treatment with the racemic mixture ASN001 at 3, 10 and 30 mg/kg PO caused 90 to 99% inhibition of testosterone; that is, the racemate was found to be more efficacious than either enantiomer dosed alone.

The effect of ASN001 and its individual enantiomers on prostate and seminal vesicle weights was evaluated after 14 days repeat oral administration in male SD rats. ASN001 caused a dose-dependent reduction of the prostate and seminal vesicle weights. ASN001 dosed at 10 mg/kg twice a day (BID) PO×14 days was found to cause a similar or greater reduction in prostate and seminal vesicle weights (24% and 48%, respectively) as compared to the more potent single enantiomer (−)ASN001 (30 and 31%, respectively), which was also dosed at 10 mg/kg BID PO×14 days. ((−)ASN001 was determined to be the more potent enantiomer in the in vitro assays; see above).

Furthermore, dosing of the racemate ASN001 at 30 mg/kg once a day (QD) PO×14 days caused greater reduction in prostate and seminal vesicle weights (29% and 43%, respectively) as compared to the single enantiomer (−)ASN001 (17 and 27%, respectively), which was dosed at 15 mg/kg once a day QD PO×14 days. (In a prior test using ASN001 at 30 mg/kg once a day (QD) PO×14 days, not made in comparison to either enantiomer, but using the same general protocol, the reduction in prostate and seminal vesicle weights was “about 50%” and “about 60%” respectively. See Example 88 of WO 2013/049559.)

In summary, 14-day oral repeat dosing of ASN001 dose-dependently reduced the weights of the prostate and seminal vesicles in male SD rats; both enantiomers of ASN001 contribute to the efficacy observed for the racemic mixture ASN001; and the racemate ASN001 is similar to or more efficacious than an equal dose of the more potent enantiomer. Chiral pharmacokinetics analysis suggests that the superior performance of the racemate ASN001 as compared to the more potent enantiomer (−)ASN001 may be due to improved disposition of (−)ASN001 in the presence of enantiomer (+)ASN001.

Advantageously the racemic mixture may be able to provide a better therapeutic index. It is possible that dosing a mixture of ASN001 and, in particular, a racemic mixture of ASN001, can provide a greater degree of testosterone synthesis suppression, at a lower dose, with less side effects. It is quite unexpected to find that the racemic mixture is potentially superior to the enantiomers therapeutically.

General procedures for the in vitro and in vivo studies discussed above have been described previously in WO 2013/049559 (published in the US as United States Patent Application Publication No. US 2013/0085148; see e.g. Examples 84-88). The inhibitory effect of compounds on human CYP17 lyase activity in vitro in human testicular microsomes (CelsisIVT, US) was determined in a similar manner to the CYP17 lyase assay in rat testicular microsomes described previously as Example 84 in WO 2013/049559 (published in the US as United States Patent Application Publication No. US 2013/0085148), except that human testicular microsomes were used instead of rat testicular microsomes.

The selectivity of ASN001 also leads to potentially desirable therapy options such as steroid-free protocols. Abiraterone, a CYP17 inhibitor, was approved by the US FDA Food and Drug Administration (FDA) in April 2011 for the treatment of patients with metastatic castration-resistant prostate cancer who have received prior chemotherapy containing docetaxel. This approval was for administration of the prodrug form abiraterone acetate (Zytiga®) at 1000 mg once daily, in combination with prednisone dosed at 5 mg twice daily (C. J. Logothetis et al., Nature Reviews Drug Discovery, 10, 573-574 (2011); E. A. Mostaghel, Cancer Management and Research, 6, 39-51 (2014)). Prednisone is co-administered with abiraterone acetate to ameliorate the rise in adrenocorticotropic hormone (ACTH) that can lead to mineralocorticoid excess.

Phase I and II trials showed that treatment with abiraterone acetate without co-administration of prednisone resulted in symptoms of mineralocorticoid excess in 50% to 80% of patients (G. Attard et al., J. Clin. Endocrinol. Metab., 97, 507-516 (2012)). Mechanistically, this is considered to be due to the relatively nonselective inhibition of the C17,20-lyase activity of CYP17 (IC₅₀=2.9 nM) as compared to the 17α-hydroxylase activity of CYP17 (IC₅₀=4 nM) (G. Attard et al., J. Clin. Endocrinol. Metab., 97, 507-516 (2012); G. A. Potter et al., J. Med. Chem., 38, 2463-2471 (1995)). As indicated in the simplified scheme (below) for androgen biosynthesis, non-selective inhibition of both CYP17 catalytic activities should result in a reduction in the synthesis of both testosterone and cortisol, and the decrease in cortisol causes a rise in ACTH with a consequent increase in 11-deoxycorticosterone (DOC) and corticosterone.

In human adrenal NCI-H295R cells, abiraterone was found to inhibit cortisol biosynthesis with IC₅₀=3 nM, whereas an IC₅₀ value could not be determined for testosterone biosynthesis due to no dose response (FDA Center for Drug Evaluation and Research, Pharmacology Review for Abiraterone, appl. no. 202379Orig1s000, http://www.accessdata.fda.gov/drugsatfda docs/nda/2011/202379Orig1s000PharmR.pdf).

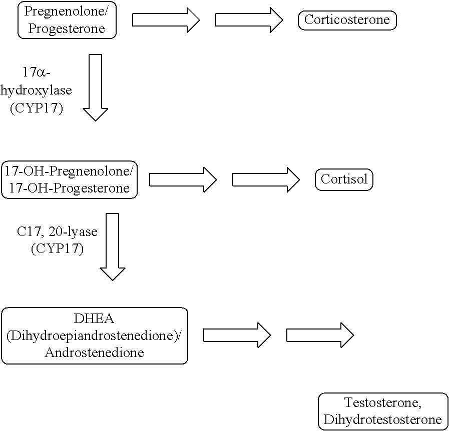

ASN001 is selective for testosterone synthesis versus cortisol synthesis, as measured in adrenal NCI-H295R cells. Inhibition in adrenal NCI-H295R cells of CYP17 C17,20-lyase activity was found in experiments to be more potent (IC_{H}=50 nM) versus inhibition of cortisol biosynthesis (IC₅₀=350 nM) (about a 7-fold difference). In comparison, abiraterone was found to inhibit C17,20-lyase activity with IC₅₀=3 nM versus inhibition of cortisol synthesis with IC₅₀<1 nM.

In further experiments using NCI-H295R cells, it was determined that ASN001 inhibited the biosynthesis of testosterone with IC₅₀=54 nM (as compared to inhibition of cortisol biosynthesis with IC₅₀=350 nM; see above), whereas corticosterone biosynthesis was inhibited with IC₅₀=2800 nM. (Note that testosterone inhibition by compound 7 (ASN001) was erroneously reported in Table 3 of WO 2013/049559 as “A,” indicating an IC₅₀<50 nM.) This finding further supports the expectation that therapy using ASN001 will selectively inhibit biosynthesis of testosterone versus biosynthesis of glucocarticoids and therefore may not require co-administration of prednisone. A summary of the data obtained in human adrenal NCI-H295R cells follows:

Human Adrenal NCI-H295R Cell Assay

| | | testosterone | cortisol | corticosterone | testosterone/cortisol |
|---|---|---|---|---|---|
| | 17,20-Lyase | synthesis | synthesis | synthesis | synthesis inhibition |
| Compound | IC₅₀ (nM) | IC₅₀ (nM) | IC₅₀ (nM) | IC₅₀ (nM) | ratio |
| ASN001 | 50 | 54 | 350 | 2800 | 6.5 |
| Abiraterone | 3 | 5 | <1 | 65 | <0.2 |

Obviously the data will vary from experiment to experiment and from procedure to procedure. More important than any specific data, however, is the fact that in these sets of experiments, ASN001 was more potent in inhibiting testosterone production than it was the production of cortisol; i.e., ASN001 was selective for inhibition of testosterone production versus inhibition of cortisol production. In comparison, abiraterone was substantially more potent as an inhibitor of testosterone than ASN001. However, abiraterone was similarly or even more potent in inhibiting cortisol as compared to inhibiting testosterone. As a result, abiraterone requires concomitant administration of a steroid with all of its consequence to the patient's qualify of life. Indeed, the longer the exposure to agents like abiraterone, with administration of prednisone the more profound the consequences of extended steroid exposure can be. Compounds such as ASN001, having far greater selectivity than abiraterone as demonstrated above, offers the possibility of better options for treating physicians and the promise of lower side effects and a better quality of life for patients.

Pharmaceutical compositions and dosage forms prepared from them may contain ASN001, either alone (“neat”) or in combination with one or more pharmaceutically acceptable carriers and/or excipients. These are pharmaceutically inert (inactive) ingredients, which, when present are together formed into dosage forms. Pharmaceutical compositions may include ASN001 as the only active ingredient or may be combined with or co-administered with other therapeutic agents as described below.

The pharmaceutical compositions, and the dosage forms produced therewith, comprise an effective amount, also referred to as a therapeutically effective amount, of ASN001, That is an amount effective to produce at least about a 30% reduction in prostate-specific an (PSA). In another embodiment, however, that the amount given should be effective to produce at least a 50% reduction in PSA. This is determined by comparison of PSA levels before taking ASN001 and PSA levels taken at a later time, such as 12 weeks after treatment began.

The dose of ASN001 useful to achieve a therapeutic effect will depend on the formulation, age, weight and sex of the patient, and the etiology and pathology of the disease or condition. It can also depend, in this instance, on whether a mixture, racemic mixture or enantiomer is used. Doses of ASN001 may also reflect the form of the ASN001 used such as whether a salt, solvate or prodrug is used.

It will be appreciated that references to a dose refers to milligrams of ASN001 free base (i.e., the neutral form of the molecule). This amount will need to be adjusted accordingly when using a salt, solvate or prodrug in view of the difference in the molecular weight and/or actual weight of the form of the active used.

In one embodiment, the therapeutically effective amount of ASN001 administered ranges from about 10 mg to about 500 mg and in another embodiment ranges from about 25 mg to about 400 mg. In another embodiment, the dose is about 50 to about 350 mg per day and in still another, it is about 100 to about 300 mg per day. This refers to the daily dose for a human patient no matter how many dosage forms it is spread over each day. These doses may be given in a single dosage form once per day, a plurality of dosage forms once per day or a plurality of dosage forms given at more than one time during each treatment day of each cycle. The dosage range per day may alternatively be: from about 10 mg/day to about 2 grams/day; from about 10 mg/day to about 1.5 grams/day; from about 10 mg/day to about 1 gram/day; from about 50 mg/day to about 900 mg/day; from about 50 mg/day to about 800 mg/day; from about 50 mg/day to about 750 mg/day; from about 50 mg/day to about 700 mg/day; from about 50 mg/day to about 600 mg/day; from about 100 mg/day to about 1 gram/day; and from about 100 mg/day to about 750 mg/day.

The therapeutically effective amount may be provided on regular schedule or cycle, i.e., daily, weekly, monthly, or yearly basis or on an irregular schedule with varying administration days, weeks, months, etc. The schedule may also vary (i.e. 2 days of administration followed by a day of placebo or rest; 21 days of administration followed by 7 days of rest; once during the first day and a rest of 21 days). Alternatively or in addition, the therapeutically effective amount to be administered may vary. In one embodiment, the therapeutically effective amount for the first daily dose is higher than the therapeutically effective amount for one or more of the subsequent daily doses. In another embodiment, the therapeutically effective amount for the first daily dose is lower than the therapeutically effective amount for one or more of the subsequent daily doses.

In one embodiment, the methods described herein contemplate administering ASN001 in a single dose given once per day, every day, for 7 consecutive days, for 10 consecutive days, for 14 consecutive days, for 21 consecutive days, for 30 consecutive days, or for 31 consecutive days. Each of the foregoing is a cycle. Cycles may be given in succession or there can be a break between cycles. Only one cycle may be necessary. Days may be skipped with skipped days added to the end of the cycle within the discretion of the treating medical professional. In still a further embodiment, ASN001 is administered every day (or substantially every day with brief interruptions and/or rest periods as determined medically necessary and appropriate) until such time that the therapy is no longer effective or cannot be tolerated by the patient.

In one embodiment, the methods described herein contemplate administering ASN001 in a divided dose given twice per day, every day (e.g. 300 mg daily dose given as 150 mg in the morning and 150 mg given approximately 12 hours later), for 7 consecutive days, for 10 consecutive days, for consecutive days, for 21 consecutive days, for 30 consecutive days, or for 31 consecutive days. Each of the foregoing is a cycle. Cycles may be given in succession or there can be a break between cycles. Only one cycle may be necessary. Days may be skipped with skipped days added to the end of the cycle within the discretion of the treating medical professional.

Doses, which may be equal or different, may be administered over various time periods including, but not limited to, about every 2 hours, about every 4 hours, about every 6 hours, about every 8 hours, about every 12 hours, about every 24 hours. The number and frequency of doses corresponding to a completed cycle of therapy will be determined according to the judgment of a health-care practitioner.

Pharmaceutical carrier(s), used synonymously herein with the term “excipients,” may be solid or liquid and oral dosage forms used may include both solid and liquid excipients. The pharmaceutical carriers must be compatible physiologically, pharmaceutically acceptable, consistent with oral administration and consistent with the intended dissolution of the ASN001 in vivo.

A variety of suitable liquid carriers is known and may be readily selected by one of skill in the art. Such carriers may include, e.g., DMSO, saline, buffered saline, hydroxypropylcyclodextrin, glycols, oils, and the like. The pharmaceutical carriers and excipients used will be and may be in dry or liquid form and must be pharmaceutically acceptable. Liquid pharmaceutical compositions are typically sterile solutions or suspensions. When liquid carriers are utilized for parenteral administration, they are desirably sterile liquids. Liquid carriers are typically utilized in preparing solutions, suspensions, emulsions, syrups, elixirs and gel-caps. In one embodiment, ASN001 is dissolved a liquid carrier. In another embodiment, ASN001 is suspended in a liquid carrier.

ASN001 may alternatively be formulated with one or more solid pharmaceutical carriers or excipients and formed into dosage forms. The composition may be, blended, granulated, encapsulated or formed into a matrix. It may be compacted into a tablet or caplet. It may be formulated as an effervescent tablet or other orally dissolvable tablet. In another embodiment, the composition may be loaded as a powder, granulate or the like into a traditional gelatin capsule. In a further embodiment, the composition may be formulated for administration as a powder, such as a powder that can be sprinkled on food for ingestion. Carrier or excipient include, without limitation, flavoring agents, lubricants, solubilizers, suspending agents, fillers, glidants, compression aids, binders, disintegrants, or encapsulating materials, pH adjusting substances, buffers, coatings, adjuvants, antioxidants, buffers, coloring agents, emulsifiers, emollients, granulating agents, ion chelators, osmo-regulators, preservatives, solubilizers, sorbents, stabilizers, sweeteners, surfactants, suspending agents, syrups, thickening agents, or viscosity regulators. See, for example, the excipients described in the “Handbook of Pharmaceutical Excipients”, 5th Edition, Eds. Rowe, Sheskey, and Owen, APhA Publications (Washington, D.C.), Dec. 14, 2005, which is incorporated herein by reference. One suitable formulation includes an effective amount of ASN001, about 25% of the formulation mixture plus about 43% of Microcrystalline Cellulose NF; about 29% Mannitol U.S.P.; about 2% Croscarmellose Sodium; and about 0.5% Magnesium Stearate in a hard gelatin capsule.

In addition to the components described above, the dosage forms and treatment methods described herein may contain one or more medications or therapeutic agents which are used to treat solid tumors. In one embodiment, the medication is a chemotherapeutic, including but not limited to cytotoxic/cytostatic agents and targeted agents such as include LHRH agonist/antagonists, androgen receptor antagonists, kinase or other enzyme inhibitors, and the like. Examples of chemotherapeutics include those recited in the “Physician's Desk Reference”, 64th Edition, Thomson Reuters, 2010, which is hereby incorporated by reference. In one embodiment, the ASN001 can be administered with other inhibitors of CYP17, such as abiraterone acetate, or with compounds that suppress testosterone production, such as LHRH agonists/antagonists. Therapeutically effective amounts of the additional medication(s) or therapeutic agents are well known to those skilled in the art. However, it is well within the professional judgment of the attending physician to determine the amount of other medication to be delivered. While in some embodiments, a steroid may be used, in other embodiments, the use of a steroid, but not other active ingredients, is specifically excluded.

The ASN001 and any other medication(s) or therapeutic agent(s) may be administered in a single dosage form. However, the present invention is not so limited. In other embodiments, the ASN001 may be administered in one or more separate oral dosage form and any other active medication is administered in one or more separate dosage forms, which need not be oral dosage forms, and need not be given on the same schedule as the ASN001. For example only, ASN001 could be administered orally once daily in the morning while another agent is given every other day in the evening and by injection. Either falls within the meaning of “concomitant” administration.

It has been found that ANS001 does not exhibit a food effect in rats and bioavailability was found to be quite high in rat and other non-clinical species. These findings suggest that there will be no food effect in human patients. Accordingly, the dosage forms, regimens and methods of administration described herein may be accomplished on humans in the fed or fasted state and with food or without. Thus, the methods described herein could include administration of a therapeutically effective amount of ASN001, as a substantially pure enantiomer, as a mixture or as a racemic mixture, which is “food indifferent” which means with or without food to a patient in a fed or fasted state. Examples include, without limitation, with food, to a patient who has consumed food in the last two hours, without food, and/or to a patient who has not eaten in the last two hours.

Also provided herein are kits or packages of pharmaceutical formulations containing dosage forms of ASN001 and/or other compositions described herein. The kits may be organized to indicate a single formulation or combination of formulations to be taken at each desired time.

Suitably, the kit contains packaging or a container with the ASN001 formulated for the desired delivery route. Suitably, the kit contains instructions on dosing and an insert regarding the active agent. Optionally, the kit may further contain instructions for monitoring circulating levels of product and materials for performing such assays including, e.g., reagents, well plates, containers, markers or labels, and the like. Such kits are readily packaged in a manner suitable for treatment of a desired indication. Kits may also include other active agents to be administered. When delivered periodically in a discontinuous fashion, a package or kit can include placebos during periods when ASN001 and/or other active ingredients are not delivered.

A number of packages or kits are known in the art for dispensing pharmaceutical agents for periodic oral use. In one embodiment, the package has indicators for each period. In another embodiment, the package is a labeled blister package, dial dispenser package, or bottle.

### EXAMPLE 1

Cell-Based CYP17 C17,20-Lyase Assay

To test the inhibitory effect of compounds on CYP17 C17,20-lyase activity in cells, human adrenocortical carcinoma H295R cells (ATCC) were used. These cells have intact testosterone synthesis pathway and are ideal to study the effect of compounds on various enzymes involved in this pathway. The cell-based CYP17 C17,20-Lyase assay was conducted as described previously as Example 85 in WO 2013/049559 (published in the US as United States Patent Application Publication No. US 2013/0085148).

Results: IC₅₀ values were obtained for ASN001 in multiple runs. The average IC₅₀ was 0.050 μM. In a similar manner, several experiments were conducted using abiraterone, and an average IC₅₀=0.0028 μM was obtained.

### EXAMPLE 2

Cell-Based Functional Assay: Testosterone Synthesis Inhibition

To understand how the inhibition of CYP17 lyase impacts testosterone synthesis, testosterone levels were measured in H295R adrenocortical carcinoma cells. This assay was conducted as described previously as Example 86 in WO 2013/049559 (published in the US as United States Patent Application Publication No. US 2013/0085148).

Results: Using ASN001 as the test sample, testosterone synthesis was inhibited with an EC₅₀ value of 0.054 μM. In a similar manner, using abiraterone as the test sample, testosterone synthesis was inhibited with an EC_{═}value of 0.005 μM.

### EXAMPLE 3

Cell-Based Functional Assay: Cortisol Synthesis Inhibition

The H295R cell based assay was used to examine effects on cortisol synthesis. The DetectX® Cortisol Immunoassay kit (Arbor Assays, Cat. No. K003-H1) measures Cortisol present in extracted dried fecal samples, serum, plasma and tissue culture media samples.

Standard Preparation
1. Label seven test tubes as #1 through #7.
2. Pipet 450 μL of Assay Buffer into tube #1 and 250 μL into tubes #2 to #7.
3. The Cortisol stock solution contains an organic solvent. Pre-rinse the pipet tip several times to ensure accurate delivery.
4. Carefully add 50 μL of the Cortisol stock solution to tube #1 and vortex completely.
5. Take 250 μL of the Cortisol solution in tube #1 and add it to tube #2 and vortex completely.
6. Repeat the serial dilutions for tubes #3 through #7.
7. The concentration of Cortisol in tubes 1 through 7 will be 5,000, 2,500, 1,250, 625, 312.5, 156.25 and 78.125 pg/mL.

Assay Protocol
1. Subculture H295R (Human Adreno Cortical Carcinoma cell line ATCC# CRL-2128) cells and seed 60,000 cells per well in a poly-d lysine plate and leave it overnight at 37° C.
2. Add 50 μl of serially diluted compounds from a 5×plate.
3. Incubate overnight at 37° C. (˜16 hours).
4. Collect media/cell culture supernatant and dilute appropriately for Cortisol estimation (1:10 diluted with assay buffer).
5. Pipet 50 μL of samples or standards into wells in the plate.
6. Pipet 75 μL of Assay Buffer into the non-specific binding (NSB) wells.
7. Pipet 50 μL of Assay Buffer into wells to act as maximum binding wells (B0 or 0 pg/mL).
8. Add 25 μL of the DetectX® Cortisol Conjugate to each well using a repeater pipet.
9. Add 25 μL of the DetectX® Cortisol Antibody to each well, except the NSB wells, using a repeater pipet.
10. Gently tap the sides of the plate to ensure adequate mixing of the reagents. Cover the plate with the plate sealer and shake at room temperature for 1 hour. If the plate is not shaken signals bound will be approximately 45% lower.
11. Aspirate the plate and wash each well 4 times with 300 μL wash buffer. Tap the plate dry on clean absorbent towels.
12. Add 100 μL of the TMB Substrate to each well, using a repeater pipet.
13. Incubate the plate at room temperature for 30 minutes without shaking.
14. Add 50 μL of the Stop Solution to each well, using a repeater or a multichannel pipet.
15. Read the optical density generated from each well in a plate reader capable of reading at 450 nm.
16. Use the plate reader's built-in 4PLC software capabilities to calculate Cortisol concentration for each sample.

Results: ASN001 showed an IC₅₀ of 0.346 μM in this assay. In a similar manner, using abiraterone as the test sample, an IC₅₀ value of 0.00068 μM was obtained.

### EXAMPLE 4

Cell-Based Functional Assay: Corticosterone Synthesis Inhibition

The H295R cell based assay was used to examine effects on corticosterone synthesis. The DetectX® Corticosterone Immunoassay kit (Arbor Assays, Cat. No. K014-H1) measures Corticosterone present in extracted dried fecal samples, serum, plasma and tissue culture media samples.

Standard Preparation
1. Label seven test tubes as #1 through #7.
2. Pipet 450 μL of Assay Buffer into tube #1 and 250 μL into tubes #2 to #7.
3. The corticosterone stock solution contains an organic solvent. Pre-rinse the pipet tip several times to ensure accurate delivery.
4. Carefully add 50 μL of the corticosterone stock solution to tube #1 and vortex completely.
5. Take 250 μL of the corticosterone solution in tube #1 and add it to tube #2 and vortex completely.
6. Repeat the serial dilutions for tubes #3 through #7.
7. The concentration of corticosterone in tubes 1 through 7 will be 5,000, 2,500, 1,250, 625, 312.5, 156.25 and 78.125 pg/mL.

Assay Protocol
1. Subculture H295R (Human Adreno Cortical Carcinoma cell line ATCC# CRL-2128) cells and seed 60,000 cells per well in a poly-d lysine plate and leave it overnight at 37° C.
2. Add 50 μl of serially diluted compounds from a 5×plate.
3. Incubate overnight at 37° C. (˜16 hours).
4. Collect media/cell culture supernatant and dilute appropriately for Corticosterone estimation (1:10 Diluted with assay buffer).
5. Pipet 50 μL of samples or standards into wells in the plate.
6. Pipet 75 μL of Assay Buffer into the non-specific binding (NSB) wells.
7. Pipet 50 μL of Assay Buffer into wells to act as maximum binding wells (Bo or 0 pg/mL).
8. Add 25 μL of the DetectX® Corticosterone Conjugate to each well using a repeater pipet.
9. Add 25 μL of the DetectX® Corticosterone Antibody to each well, except the NSB wells, using a repeater pipet.
10. Gently tap the sides of the plate to ensure adequate mixing of the reagents. Cover the plate with the plate sealer and shake at room temperature for 1 hour. If the plate is not shaken signals bound will be approximately 45% lower.
11. Aspirate the plate and wash each well 4 times with 300 μL wash buffer. Tap the plate dry on clean absorbent towels.
12. Add 100 μL of the TMB Substrate to each well, using a repeater pipet.
13. Incubate the plate at room temperature for 30 minutes without shaking.
14. Add 50 μL of the Stop Solution to each well, using a repeater or a multichannel pipet.
15. Read the optical density generated from each well in a plate reader capable of reading at 450 nm.
16. Use the plate reader's built-in 4PLC software capabilities to calculate Corticosterone concentration for each sample.

Results: ASN001 showed an IC₅₀ of 2.8 μM in this assay. In a similar manner, using abiraterone as the test sample, IC₅₀=0.065 μM was obtained.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of treating a human patient having prostate cancer comprising: administering orally to a human patient in need thereof a therapeutically effective amount of racemic ASN001 ranging from about 10 mg to about 2 grams in no more than 2 doses per day for at least 14 consecutive days.

2. The method of claim 1, wherein the amount of ASN001 administered daily ranges from about 25 to about 400 mg.

3. The method of claim 2, wherein the amount of ASN001 administered daily ranges from about 50 to about 300 mg.

4. The method of claim 1, wherein the ASN001 is administered in a single dose per day.

5. The method of claim 4, wherein the amount of ASN001 administered daily ranges from about 25 to about 400 mg.

6. The method of claim 5, wherein the amount of ASN001 administered daily ranges from about 50 to about 300 mg.

7. The method of claim 1, wherein the human patient has castration-resistant prostate cancer.

8. The method of claim 7, wherein the amount of ASN001 administered daily ranges from about 25 to about 400 mg.

9. The method of claim 8, wherein the amount of ASN001 administered daily ranges from about 50 to about 300 mg.

10. The method of claim 1, wherein the racemic ASN001 is administered for at least 30 consecutive days.

11. The method of claim 1, in which the human patient is not treated with concomitant of a steroid.

12. The method of claim 1, in which the human patient is not treated concomitantly with prednisone.

13. The method of claim 1, wherein the ASN001 is in the form of the free base.

14. A method of treating a cancer susceptible to testosterone deprivation in a human patient suffering from same comprising: administering orally to a human patient in need thereof a chemotherapeutic regimen consisting essentially of a therapeutically effective amount of ASN001 ranging from about 10 mg to about 2 grams daily in 1 to 4 doses per day for at least 14 consecutive days.

15. The method of claim 14, wherein the amount of ASN001 administered daily ranges from about 25 to about 400 mg.

16. The method of claim 15, wherein the amount of ASN001 administered daily ranges from about 50 to about 300 mg.

17. The method of claim 14, wherein the ASN001 is administered in no more than 2 doses per day.

18. The method of claim 17, wherein the ASN001 is administered in a single dose per day.

19. The method of claim 18, wherein the amount of ASN001 administered daily ranges from about 25 to about 400 mg.

20. The method of claim 14, wherein the administration of ASN001 is food indifferent.

21. The method of claim 14, in which the ASN001 is a substantially pure optical isomer.

22. The method of claim 14, wherein the chemotherapeutic regimen is administered for at least 30 days.

23. A daily chemotherapeutic regimen of ASN001 suitable for administration to a human patient comprising: oral dosage forms containing from about 10 mg to about 2 grams of racemic ASN001 divided into from 1 to 4 daily doses.

24. The daily regimen of claim 23, wherein the amount of ASN001 ranges from about 25 to about 400 mg divided in no more than 2 doses per day.

25. The daily regimen of claim 24, wherein the amount of ASN001 ranges from about 50 to about 300 mg divided into no more than 2 doses per day.

26. A solid oral dosage form comprising: about 25 to about 400 mg of racemic ASN001 and at least one excipient.

27. A solid oral dosage form comprising: about 25 to about 400 mg of a mixture of enantiomers ASN001 and at least one excipient.

28. A solid oral dosage form comprising: about 25 to about 400 mg of a substantially pure enantiomer of ASN001 and at least one excipient.

29. The solid oral dosage form of claim 26 which is a tablet, gel capsule, caplet or liquid gel capsule.
